(19) United States
(12) Patent Application Publication
(10) Pub. No.: US 2019/0180637 A1
(43) Pub. Date: Jun. 13, 2019

(54) Virtually Resilient Simulator

(71) Applicant: The Regents of the University of Colorado, a body corporate, Denver, CO (US)

(72) Inventor: Meredith Mealer, Louisville, CO (US)

(21) Appl. No.: 16/215,149

(22) Filed: Dec. 10, 2018

Related U.S. Application Data
(60) Provisional application No. 62/596,608, filed on Dec. 8, 2017.

(51) Int. Cl. G09B 5/04; G06F 3/01

(52) U.S. Cl. CPC G09B 5/04; G06F 3/016; G06F 3/014

(57) ABSTRACT

Various embodiments of the present technology generally relate to virtual and augmented reality simulations. More specifically, some embodiments of the present technology generally relate to virtual and augmented reality simulations to improve resiliency for healthcare clinicians. Some embodiments provide for a VR training system to allow a realistic, immersive and safe environment for students or other trainees to encounter traumatic experiences. Exposure to trauma in a safe environment offers the opportunity for new and seasoned individual to build resilient skills through cognitive flexibility and reframing thus alleviating symptoms of burnout, PTSD, anxiety and depression that is caused by the work environment and critical encounters. The participant can be the ‘player’ within simulation hosting multiple virtual characters and/or multiple other ‘players’ for team building and organizational resilience.

### CROSS REFERENCE TO RELATED APPLICATIONS

This application claims priority to U.S. Provisional Application Ser. No. 62/596,608 filed Dec. 8, 2017, which is incorporated herein by reference in its entirety for all purposes.

### TECHNICAL FIELD

Various embodiments of the present technology generally relate to virtual and augmented reality simulations. More specifically, some embodiments of the present technology generally relate to virtual and augmented reality simulations to improve resiliency for healthcare clinicians, military members, and other individuals.

### BACKGROUND

Acute care institutions and specialty hospitals with intensive care units can be stressful environments. The high acuity and stress areas of acute care institutions have the highest prevalence of anxiety, depression, posttraumatic stress disorder and burnout syndrome. These areas also have higher turnover rates than other healthcare institutions. Resilience to these negative effects can be taught and learned by the medical professionals. Unfortunately, most of the focus of traditional training and education is around teaching the mechanics of various medical procedures.

### SUMMARY

Various embodiments of the present technology generally relate to virtual and augmented reality simulations. More specifically, some embodiments of the present technology generally relate to virtual and augmented reality simulations to improve resiliency for healthcare clinicians, military members, and other individuals. In some embodiments, a method for operating a virtual reality environment can include generating, using an event simulator, a virtually reality scenario. The virtual reality scenario may be based on objectives or desired outcome set by an operator or an automated analysis of historical ability of a user. A control system can translate the virtual reality scenario into a set of commands for one or more devices (e.g., physical equipment, haptic glove, sent synthesizer, etc.). The user may be wearing a virtual reality headset to visually display the virtually reality scenario and a haptic glove to provide tactile feedback. The set of commands can be transmitted to these devices to create a realistic experience for the user. The one or more devices may be located within a training room (e.g., an emergency room, a hospital room, an operating room, etc.). The training room may be setup with various sensor (e.g., camera, thermal imaging, microphone, pressure sensors, heart rate monitor, medical equipment, etc.) to monitor the actions and responses of the user.

In some embodiments, the one or more sensors within the training room can transmit indications of user responses to the virtual reality scenario which can be analyzed, using an artificial intelligence system. The artificial intelligence system can dynamically update scenes within the virtual reality scenario. In some embodiments, the user may be a first user of multiple users. The artificial intelligence system can analyze the dialogue between the multiple users and updating scenes within the virtual reality scenario based on results from the artificial intelligence system analysis.

Some embodiments provide for a system comprising a training room having physical equipment (e.g., a dummy, a bed, one or more IV systems, a curtain, a sink, a ventilator machine, etc.) and sensors capable of monitoring and recording interactions from a user. The system may also include a database of user scenarios that include dialogue between individuals, medical equipment parameters, and physiological parameters of a patient. These user scenarios may have been recorded from live hospital interactions or other training sessions. An artificial intelligence system may also be used to ingest the user scenarios stored in the database, and upon receiving input signals from the physical equipment and sensors within the training room generate updates to a dynamically changing virtual reality scenario.

In some embodiments, the system may include a virtual reality event simulator configured to receive the updates to the dynamically changing virtual reality scenario from the artificial intelligence system and generate a sequence of scene to be presented to the user. The system may also have a control system to receive the updates to the dynamically changing virtual reality scenario from the artificial intelligence system and the scenes from the virtual reality event simulator. Using this information, the control system can translate the updates to commands for the medial equipment and sensors to create a unique experience for the user. The user may be waring a virtual reality headset and the control system can be configured to generate updated images that can be presented to the user via the virtual reality headset. The system may also include a scent synthesizer to generate one or more smells (e.g., blood, saline solution, fluids, etc.) as indicated by the control system.

Embodiments of the present invention also include computer-readable storage media containing sets of instructions to cause one or more processors to perform the methods, variations of the methods, and other operations described herein.

While multiple embodiments are disclosed, still other embodiments of the present invention will become apparent to those skilled in the art from the following detailed description, which shows and describes illustrative embodiments of the invention. As will be realized, the invention is capable of modifications in various aspects, all without departing from the scope of the present invention. Accordingly, the drawings and detailed description are to be regarded as illustrative in nature and not restrictive.

### BRIEF DESCRIPTION OF THE DRAWINGS

Embodiments of the present technology will be described and explained through the use of the accompanying drawings.

FIG. 1 illustrates an example of an environment in which some embodiments of the present technology may be utilized.

FIG. 2 illustrates a set of components within a device according to one or more embodiments of the present technology.

FIG. 3 is a flowchart illustrating a set of operations for operating a simulation and training platform in accordance with some embodiments of the present technology.

FIG. 4 is a flowchart illustrating a set of operations for directing a simulation in accordance with one or more embodiments of the present technology.

FIG. 5 is a sequence diagram illustrating an example of the data flow between various components of a simulation platform according to various embodiments of the present technology.

FIG. 6 illustrates sample images of participant view from a head mounted display (HMD) in accordance with some embodiments of the present technology.

FIG. 7 illustrates an example of a graphical user interface that may be used in one or more embodiments of the present technology.

FIG. 8 illustrates an example of a graphical user interface that may be part of a facilitator dashboard according to various embodiments of the present technology.

FIG. 9 is a block diagram illustrating an example machine representing the computer systemization of the simulation system that may be used in some embodiments of the present technology.

The drawings have not necessarily been drawn to scale. Similarly, some components and/or operations may be separated into different blocks or combined into a single block for the purposes of discussion of some of the embodiments of the present technology. Moreover, while the technology is amenable to various modifications and alternative forms, specific embodiments have been shown by way of example in the drawings and are described in detail below. The intention, however, is not to limit the technology to the particular embodiments described. On the contrary, the technology is intended to cover all modifications, equivalents, and alternatives falling within the scope of the technology as defined by the appended claims.

### DETAILED DESCRIPTION

Various embodiments of the present technology generally relate to virtual and augmented reality simulations. More specifically, some embodiments of the present technology generally relate to virtual and augmented reality simulations to improve resiliency for healthcare clinicians, military members, law enforcement, and other individuals regularly placed in stressful and traumatic situations.

Various embodiments provide a virtual reality (VR) environment combining a 3D environment with artificial intelligence (AI) technology. The VR environment can be an immersive environment involving visual, auditory, olfactory, kinesthetic, and/or haptic/tactile feedback. The VR environment can host a variety of scenarios including various scenarios that contribute to psychological distress and symptoms associated with burnout syndrome, anxiety, depression and posttraumatic stress disorder. As such, some embodiments of the VR system offer the opportunity for training and prevention.

Some embodiments utilize a tactile/haptic glove or other covering to allow the clinician to have the sensation of touch common to the scenario. For example, in a health care scenario the clinician or medical professions may feel, through the use of the tactile/haptic glove or other covering, the cracking of ribs during cardiopulmonary resuscitation, pulling central venous catheters, removing chest tubes, defibrillator discharge and handling the body during post-mortem care.

The simulation system used by various embodiments can enhance learning by invoking affective experiences that are consistent with emotions experienced when practicing skills outside the clinical context. Some embodiments provide several key advantages over the standard instructional approaches (e.g., written practice assignments or worksheets), but also role-play and video vignettes. Specifically, the sensory-rich immersive environments of VR (e.g., virtual characters, visual ambience, directional audio, culturally specific content) can provide a realistic avenue for behavior rehearsal in a controlled environment. Further, VR environments provided by some embodiments can provide a standardized setting that can be replicated to deliver the practice opportunity in a systematic manner. In addition, a graduated application of concepts can be delivered so that the experience is challenging, but not overwhelming. Finally, incorporating interactivity and serious gaming mechanics introduces a compelling and engaging experience that motivates and supports behavior change.

Virtual characters used within various embodiments go through an additional process that allows them to move in a naturalistic way called rigging. The rigging allows the facial expressions, body movements, and lip sync to be matched. Voice recordings to match the virtual character are also created and synced to the animations. For example, a 3D environment can be created (e.g., using Unity). Integration can be ensured (e.g., using the Pullstring® Platform) and communication of information with a server and the AI engine to produce a response quickly (e.g., within a millisecond). The AI engine can use the transcripts of the dialogues to identify any adjustments to the dialogue tables. This process increases the accuracy of the virtual character's responses. Various visual assets, environments, and/or character can be created within the environment (e.g., using Autodesk Maya/Mudbox and the Adobe Creative Suite).

Various embodiments of the present technology provide for a wide range of technical effects, advantages, and/or improvements to computing systems and components. For example, various embodiments include one or more of the following technical effects, advantages, and/or improvements: 1) intelligent presentation of scoped content based on user interactions to efficiently introduce complicated scenarios that often results in psychological distress for participants; 2) cross-platform integration of machine learning and virtual reality to present realistic and dynamic training scenarios; 3) VR system with emotional engagement that provides fear structure context-related stimuli and content matching; 4) proactive and gradual training based on user experience and needed skill level; 5) use of unconventional and non-routine computer operations to contextually provide coping tool for user so that they may better handle real-life scenarios; 6) integrated use of scaffolding learning techniques to teach stressful and complex life saving techniques; 7) changing the manner in which a computing system monitors and reacts to training-based gestures, speech, planning, and problem solving; 8) complex integration of complex experiences within an educational and prevention tool; and/or 9) changing the manner in which a computing system reacts to user interactions and feedback.

In the following description, for the purposes of explanation, numerous specific details are set forth in order to provide a thorough understanding of embodiments of the present technology. It will be apparent, however, to one skilled in the art that embodiments of the present technology may be practiced without some of these specific details. While, for convenience, embodiments of the present technology are described with reference to a VR training environment to cultivate resiliency in healthcare professionals, military members, law enforcement, and other individuals regularly placed in stressful and traumatic situations, embodiments of the present technology are equally applicable to various other training needs.

The techniques introduced here can be embodied as special-purpose hardware (e.g., circuitry), as programmable circuitry appropriately programmed with software and/or firmware, or as a combination of special-purpose and programmable circuitry. Hence, embodiments may include a machine-readable medium having stored thereon instructions which may be used to program a computer (or other electronic devices) to perform a process. The machine-readable medium may include, but is not limited to, floppy diskettes, optical disks, compact disc read-only memories (CD-ROMs), magneto-optical disks, ROMs, random access memories (RAMs), erasable programmable read-only memories (EPROMs), electrically erasable programmable read-only memories (EEPROMs), magnetic or optical cards, flash memory, or other type of media/machine-readable medium suitable for storing electronic instructions.

The phrases “in some embodiments,” “according to some embodiments,” “in the embodiments shown,” “in other embodiments,” and the like generally mean the particular feature, structure, or characteristic following the phrase is included in at least one implementation of the present technology, and may be included in more than one implementation. In addition, such phrases do not necessarily refer to the same embodiments or different embodiments.

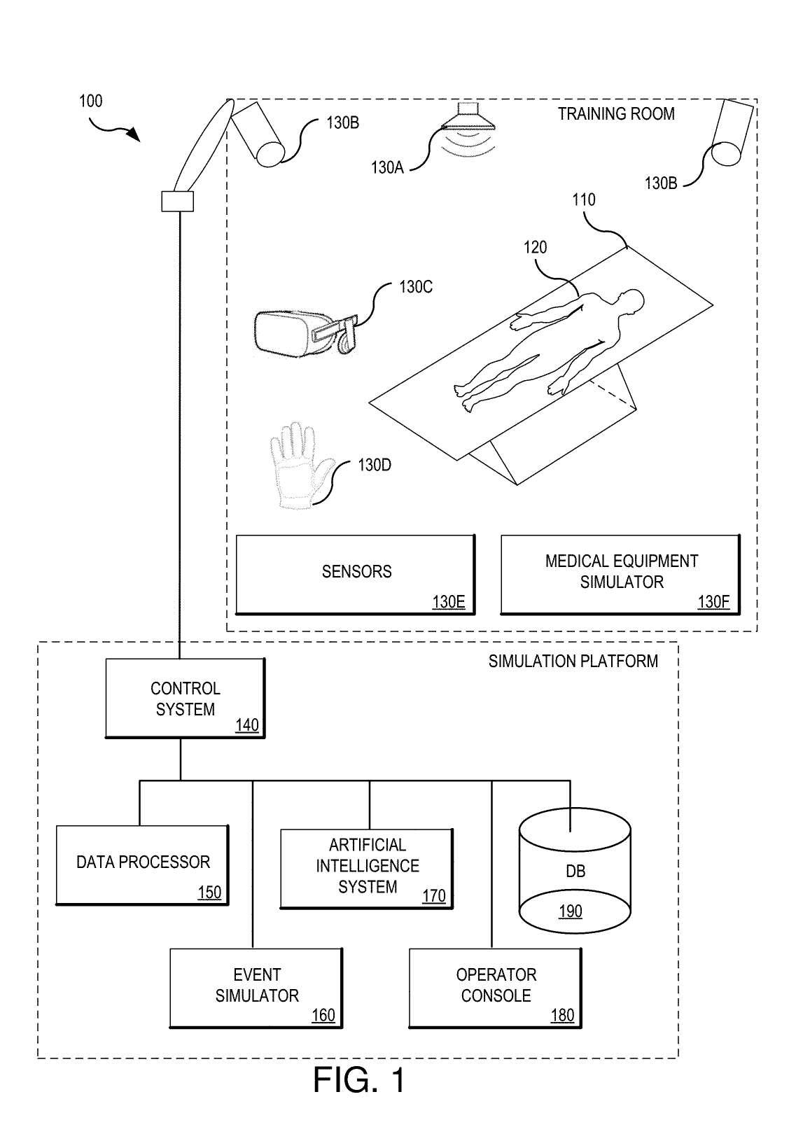

FIG. 1 illustrates an example of an environment 100 in which some embodiments of the present technology may be utilized. As illustrated in FIG. 1, environment 100 may include a training room having a table 110, training dummy 120, and various other devices 130A-103F for creating simulated environments and/or monitoring trainee interactions. For example, devices 130A-130F may include speakers 130A for creating various sound effects, cameras 130B, virtual reality (VR) headsets worn by trainees, haptic gloves 130D, sensors 130E (e.g., for measuring movement, speech, facial expressions, gestures, location, and/or physiological responses of the trainee), and various medical equipment simulators. These devices can communicate with a simulation platform to create various simulations. In some embodiments, the simulations can be modelled off real-life scenarios recorded previously, fictional scenarios specifically designed to test specific skills, evoke certain reactions (e.g., emotional reactions), and the like, and/or a combination of real-life and fictional scenarios.

The components in the training room may be connected to the platform using a communications network (not shown). As such, the components 110-130, can include network communication components that enable the mobile devices to communicate with the simulation platform or other portable electronic devices by transmitting and receiving wired signals or wireless signals using licensed, semi-licensed or unlicensed spectrum over communications network. In some cases, the communication network may be comprised of multiple networks, even multiple heterogeneous networks, such as one or more border networks, voice networks, broadband networks, service provider networks, Internet Service Provider (ISP) networks, and/or Public Switched Telephone Networks (PSTNs), interconnected via gateways operable to facilitate communications between and among the various networks.

In some embodiments, the simulation platform may interact with multiple training rooms each having one or more trainees to create a common simulated experience. An operator can use operator console 180 to command and select various scenarios within the training room. These commands may be used by event simulator to create a scenario or changes to the scenario to be implemented. In some embodiments, machine learning may be used to build custom scenarios, in response to the selected scenarios, from a large database of real-life situations that have been recorded and stored in database 190. For example, the commands from the operator may indicate high-level situational goals, learning tasks/goals, or specific actions. The goals can then be ingested by AI system 170 along with scenarios recorded in database 190. The AI system can then formulate specific environmental parameters (e.g., scripts for participants, noises, readings from instrumentations, etc.) which are implemented at the appropriate time by event simulator 160. In some embodiments, the environmental parameters may have a probabilistic range or probabilistic likelihood of occurring. As a result, the scenarios are never identical and more like real-life situations.

The scenarios can be sent and to control system 140 to send commands to devices 110-130 to create a simulated environment. Devices 110-130 can send information back to control system 140 which can then be used to update the simulation. Examples of the information sent back by devices 110-130 can include, but are not limited to, speech, actions (e.g., dials turned, buttons pushed, force applied to training dummy 120), and the like. This information can be processed by data processor 150 and AI system 170. The processed information can then be used by event simulator 160 and/or AI system 170 to update the current state of the scenario. As such, the decisions and actions made by the participants during the training directly effect the unfolding of events and/or outcome.

Some embodiments can create a variety of experiences and scenarios. For example, a virtually resilient experience created by the environment in FIG. 1 can be a virtual reality environment that will allow a healthcare provider to experience difficult and traumatic events that are common in the acute care setting, in a controlled and safe environment. The virtually resilient environment can be used as an educational training and preparedness device but also as a virtual exposure environment for treatment and prevention of psychological disorders common in the acute care health provider such as burnout syndrome and PTSD. This interactive environment can allow for visual, tactile, auditory and olfactory immersion by the clinician. The interactive environment can also simulate death (traumatic or otherwise) and trauma such as being able to feel ribs crack during chest compressions and the smell and sight of blood as their patient fails to respond to resuscitation efforts.

In accordance with various embodiments, the experience of the trainee in the simulator and other real-life situations can be monitored and recorded. By analyzing the relative experience of the trainee, the trainee can be guided with various support during the learning process which is tailored to the needs of the trainee to allow for specific learning goals to be accomplished without overwhelming the trainee.

Various embodiments can create a virtual and interactive environment for a complex critically ill patient that will undergo an unsuccessful cardiopulmonary resuscitation effort. The patient can be mechanically intubated and ventilated, sedated and will be receiving life-sustaining support for multi-organ dysfunction. The patient scenario will immerse the user in an environment that will accentuate the sounds of the various machines, the smell of blood and weeping fluid and the visual effects of a critically ill patient with severe sepsis, receiving vasopressor support, continuous renal replacement therapy (CRRT) and the sequelae of disseminated intravascular coagulation. In addition, the user may perform CPR and be able to feel the sensation of ribs cracking. During post-mortem care, the user may be able to feel the sensations of removing the endotracheal tube and chest tube, which are quite distinct. This method of exposure and interaction will allow for a safe and realistic curriculum/teaching experience but will also support behavior change and mitigation of psychological symptoms through habituation and extinction of the fear response. These changes can be guided by an operator and/or AI system 170 itself.

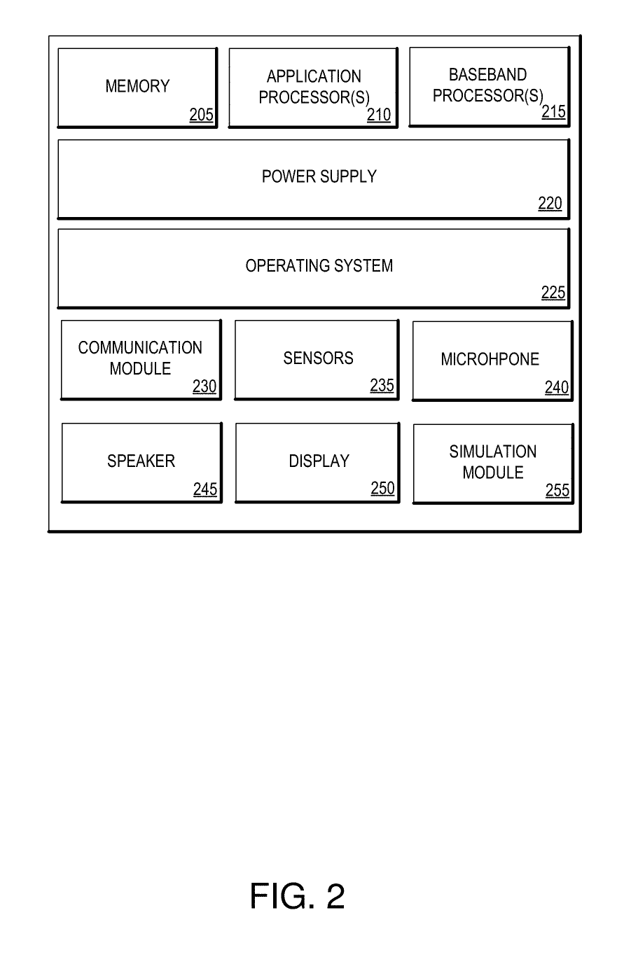

FIG. 2 illustrates a set of components within a device 110-130 according to one or more embodiments of the present technology. As shown in FIG. 2, device 110-130 may include memory 205 (e.g., volatile memory and/or nonvolatile memory), one or more processors 210-215, power supply 220 (e.g., a battery), operating system 225, communication module 230, sensors 235, microphone 240, speakers 245, display 250, simulation modules 255, and/or additional components (e.g., audio interfaces, keypads or keyboards, and other input and/or output interfaces).

Memory 205 can be any device, mechanism, or populated data structure used for storing information. In accordance with some embodiments of the present technology, memory 205 can encompass any type of, but is not limited to, volatile memory, nonvolatile memory and dynamic memory. For example, memory 205 can be random access memory, memory storage devices, optical memory devices, media magnetic media, floppy disks, magnetic tapes, hard drives, SDRAM, RDRAM, DDR RAM, erasable programmable read-only memories (EPROMs), electrically erasable programmable read-only memories (EEPROMs), compact disks, DVDs, and/or the like. In accordance with some embodiments, memory 205 may include one or more disk drives, flash drives, one or more databases, one or more tables, one or more files, local cache memories, processor cache memories, relational databases, flat databases, and/or the like. In addition, those of ordinary skill in the art will appreciate many additional devices and techniques for storing information which can be used as memory 205.

Memory 205 may be used to store instructions for running one or more applications or modules on processor(s) 210-215. For example, memory 205 could be used in one or more embodiments to house all or some of the instructions needed to execute the functionality or controlling operating system 225, communication module 230, sensors 235, microphone 240, speakers 245, display 250, simulation modules 255, and/or additional components. Operating system 225 can provide a software package that is capable of managing the hardware resources of device 110-130. Operating system 225 can also provide common services for software applications running on processor(s) 210-215.

Processors 210-215 are the main processors of device 110-130 which may include application processors, baseband processors, various coprocessors, and other dedicated processors for operating device 110-130. For example, application processor 210 can provide the processing power to support software applications, memory management, graphics processing, and multimedia. Application processor 210 may be communicably coupled with memory 205 and configured to run the operating system 225, the user interface, and the applications stored on memory 205 or data storage component (not shown). Baseband processor 215 may be configured to perform signal processing and implement/manage real-time radio transmission operations of device 110-130. These processors along with the other components may be powered by power supply 220. The volatile and nonvolatile memories found in various embodiments may include storage media for storing information such as processor-readable instructions, data structures, program modules, or other data. Some examples of information that may be stored include basic input/output systems (BIOS), operating systems, and applications.

Communication module 230 can enable the device to communicate with other devices, servers, or platforms by transmitting and receiving wireless signals using licensed, semi-licensed or unlicensed spectrum over a telecommunications network. These signals can include location information, physiological information, and other data from sensors 235. Microphone 240 can be used to identify sounds within the room while speaker 245 can create sounds mimicking the simulated environment. Some devices (e.g., VR headset 130C) may include a display 250 for displaying visual information to the trainee. Simulation module 255 can control the device according to the instructions received by the simulation platform. In some embodiments, the simulation platform may provide high level instructions which simulation module 255 is responsible for translating and implementing on the device (e.g., tactile feel created in glove 130D).

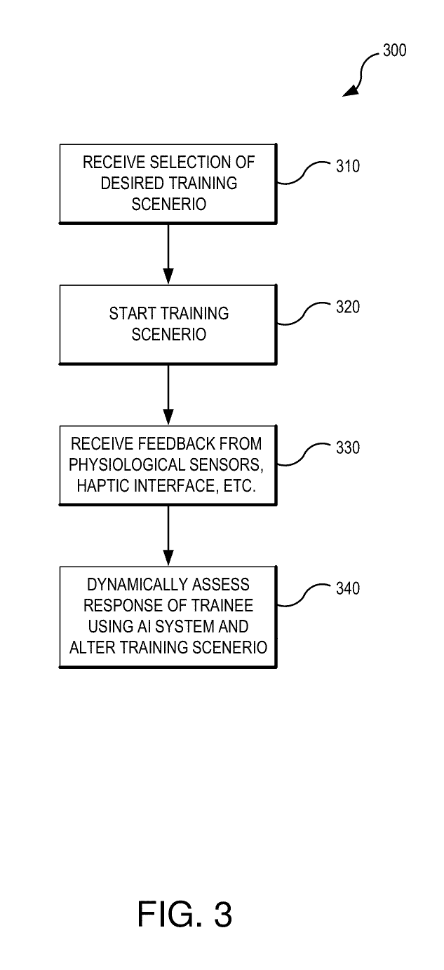

FIG. 3 is a flowchart illustrating a set of operations 300 for operating a simulation and training platform in accordance with some embodiments of the present technology. As illustrated in FIG. 3, receiving operation 310 receives a selection of desired training scenario. The training scenario can be retrieved from a database or automatically generated based on inputs from an operator of the platform.

For example, the training scenario can include a 45 year old male patient admitted with severe sepsis that presents with hypotension, respiratory failure and renal failure. Within 48 hours of admission, renal failure worsens requiring continuous renal replacement therapy (CRRT) and patient develops disseminated intravascular coagulation (DIC). The patient is sedated, on the ventilator, receiving vasopressor support for blood pressure and CRRT for renal failure. The characters could include the following: 1) the patient-with massive edema, hemorrhagic necrosis, oozing of blood from orifices; 2) the outgoing shift nurse giving report; 3) respiratory therapist; and 4) additional nurse on duty. The user (i.e., the player) could be the nurse coming on duty. The location may be an intensive care unit and equipment in the scenario can include a CRRT machine, an EKG monitor, a Foley Catheter, IV drips for hypotension, IV drips for sedation, IV fluids and other medications, blood products, multiple IV pumps and poles, crash cart with defibrillator, intubation kit and ACLS drugs, face shields and gowns, and ambu bag for manual ventilation.

The simulation can include various olfactory and auditory outputs. For example, these can include blood, edema-weeping fluids, ventilator, EKG, CRRT machine, IV pumps beeping, overhead chatter in hospital, defibrillator charging, and the like. The various characters may also be speaking. For example, character #2 may be giving report on the patient to the oncoming nurse (Player). Patient neurological signals can create a patient that is sedated and nonresponsive, pupils are 4 mm and react to light, and sedated with fentanyl and versed-taper as needed. CV can indicate that the patient continues to be hypotensive with BP 80/50, having difficulty keeping BP stable, will need to hang Levophed when it arrives from the pharmacy, currently receiving the first of 2 units of blood and IVF's at 250 cc/hr. Dopamine can be infusing at 20 mcg. Currently, the patient may be in sinus tachycardia, rate 130-140. Febrile 38.5 degrees Celsius. generalized 3-4+edema, significant orbital edema, weeping fluids and blood from eyes, nose, mouth and other orifices, radial pulses 1+ and weak, and/or pedal pulses present with a doppler. Heart sounds are muffled. Extremities are cool to touch.

Respiratory simulations can include the patient being intubated with #8.0 endotracheal tube (ETT), assist Control (AC) ventilation rate 12, tidal volume 650, 80% FiO2, 15 PEEP-titrate to keep O2 sats >90%, lungs coarse bilaterally, decreased breath sounds in the bases and, suctioning every 2 hours with thick yellowish/brown secretions. GI simulations can show a nasogastric tube is in place to intermittent suction-dark green secretions, abdomen is distended but soft, bowel sounds are present, and no stools. GU simulations can show foley in place with no output and a CRRT. This is example is illustrative of one of many scenarios and can be visualized and felt through various devices such as, but not limited to a VR display, a haptic interface (e.g., a glove), smell generators, speakers, and the like.

Initiation operation 320 starts the training scenario. This can include sending various commands to devices to create the simulated environment. The user can respond to the environment and any response data can be sent back to the simulation and training platform which are acknowledged during receiving operation 330. Assessment operation 340 can use the feedback from the response data to dynamically assess the response of the trainee (e.g., using an artificial intelligence system) and update the training scenario.

For example, during the scenario the user can interact with the other characters. In the example described above, character #2 can take the player into the ICU room to show her the CRRT settings. They both cam put on gowns and face shields. Upon arrival in the room, patient's heart rate drops from 135 to 70 and blood pressure through the arterial line fails to read a measurement. The player can feel for a carotid pulse and does not find one. Character #1 can be in pulseless electrical activity. Character #2 cam yell to the nursing station to call a code.

Character #2 can ask the player to start chest compressions. The player can begin chest compressions and feels ribs cracking as they are delivered. Character #3 and #4 arrive in the room. Character #4 can have the crash cart with the defibrillator. Character #3 can remove Character #1 from the ventilator and starts hyperventilating the patient with an ambu bag at 100%. Character #2 can assume the role of managing the code and writing the drugs and times as appropriate. Character #4 can be administering ACLS drugs per the right subclavian catheter in place (this will get more specific as far as what drugs are being administered based on the heart rhythm).

After 5 minutes of resuscitation, Character #1 can develop ventricular tachycardia on the monitor. Character #2 calls for defibrillation with the biphasic defibrillator with 3 stacked shocks at 120 J-150 J-200 J. Character #2 can charge the defibrillator, once charged, the player stops chest compressions and puts the defibrillator paddles on Character #1, the player can clear the patient, Character #3 can stop bagging Character #1 and clears the bed, the layer can call all clear and delivers the first shock, defibrillator recharged by Character #2 and repeated at 150 J and 200 J.

The simulation can show that there is a reversal of the ventricular tachycardia with electrical activity at a rate of 56 bpm but no pulse. The player can restart chest compressions (when this has the team module, if the player gets tired from the chest compressions, s/he can call for a switch and one of the other teammates will take over while the player doing chest compressions moves to a different role) and Character #3 resumes bagging and Character #4 continues administering ACLS drugs as instructed by Character #2. The ACLS drug protocol can resume and last for another 10 minutes.

After a total of fifteen minutes and 3-stacked shocks, Character #1 does not regain a pulse or blood pressure and he is pronounced dead. Post-mortem care can be done by the player only. Characters #2, #3 and #4 can leave the room. The player can remove the ETT, the subclavian catheter, the jugular catheter used for CRRT, peripheral IV lines and Foley catheter. The player can clean the blood and fluid from Character #1. There is a distinct feel when removing ETT's and the simulator would allow the player to ‘feel’ and ‘hear’ these events along with a continuous smell of blood and weeping fluids throughout the experience.

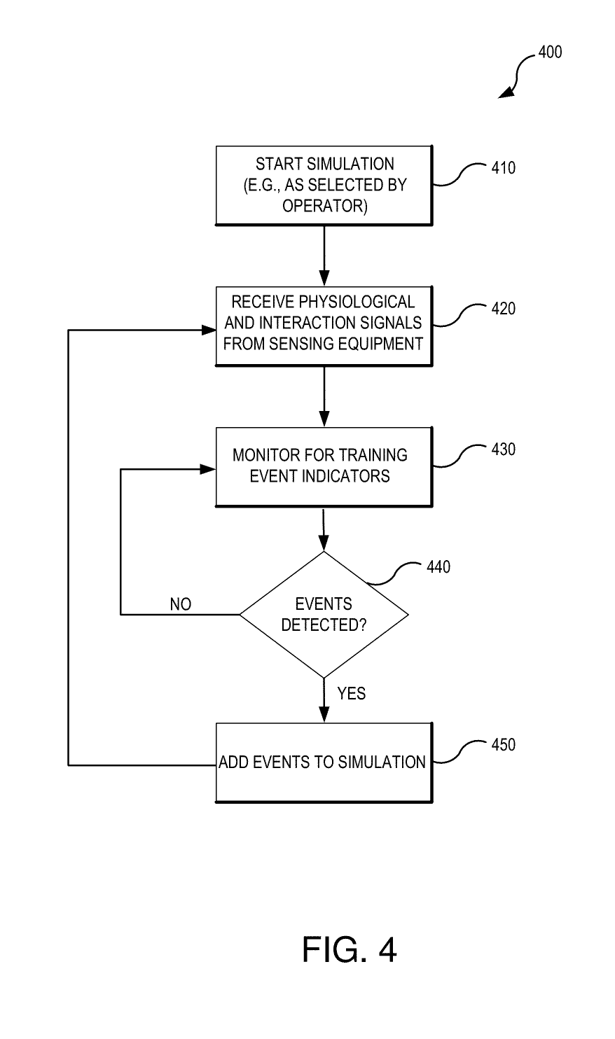

FIG. 4 is flowchart illustrating a set of operations 400 for directing a simulation in accordance with one or more embodiments of the present technology. As illustrated in FIG. 4, initiation operation 410 starts the simulation. As the user interacts to the simulation, various devices can transmit physiological and interaction information which can be transmitted to and received at the training and simulation platform during receiving operation 420. Monitoring operation 430 can analyze the information to identify any training event indicators. When identification operation 440 determines that no event indicators has been detected, identification operation 440 can branch to monitoring operation 430 to continue to monitor for the training event indicators. When identification operation 440 determines that an event indicator has been detected, identification operation 440 can branch to addition operation 450 where the scenario can be modified accordingly.

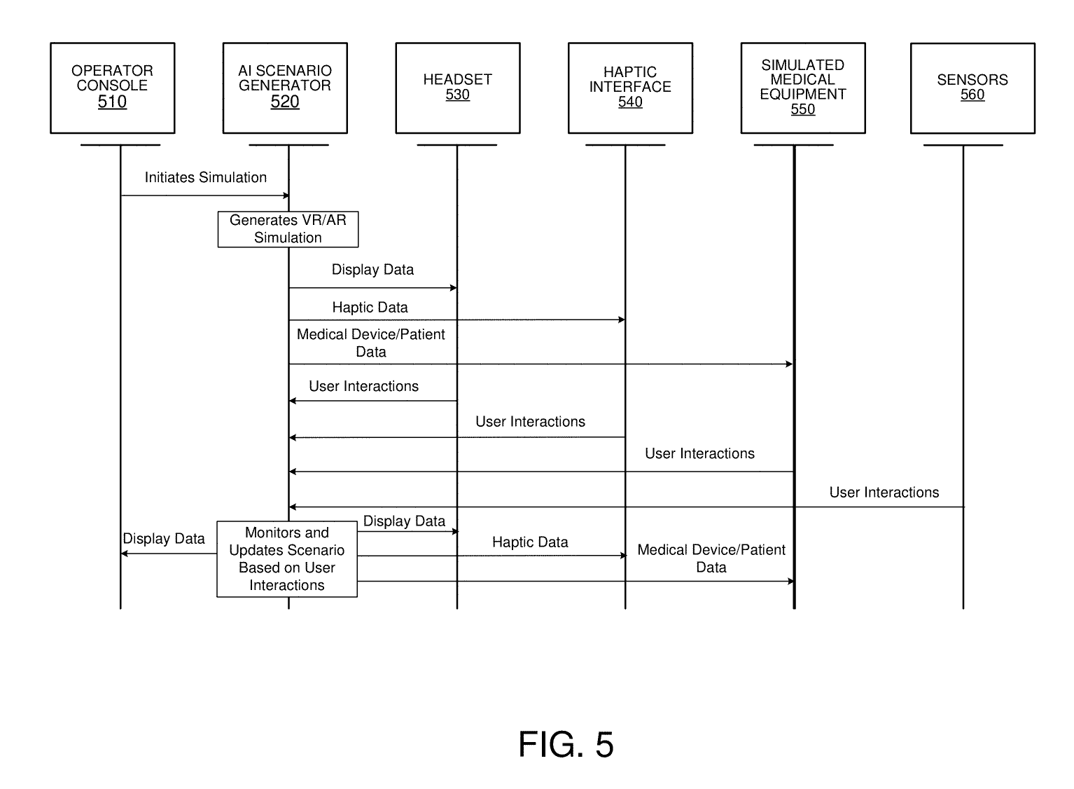

FIG. 5 is a sequence diagram illustrating an example of the data flow between various components of a simulation platform according to various embodiments of the present technology. As illustrated in FIG. 5, operating console 510 can be used to select and initiate a simulation. AI scenario generator 520 can generate a VR/AR simulation based on the selected parameters identified by the user of operation console 510 and available equipment in the training room. For example, in accordance with some embodiments, AI scenario generator 520 can generate display data, haptic data, medical device/patient data, and/or other environmental parameters. This information can be transmitted to the corresponding components (e.g., headset 530, haptic interface 540, simulated medical equipment 550, and the like) within the training room.

As the simulations progresses, data from the components (e.g., haptic interface 540, simulated medical equipment 550, etc.) and sensors 560 (e.g., cameras, microphones, etc.) within the room can be transmitted back to the AI scenario generator 520 to provide feedback and information that can be used to dynamically update the simulation with the user interactions (or lack thereof). AI scenario generator 520 can analyze the responses and generate scoring and notifications (e.g., of missed actions, areas for improvement, etc.) that can be transmitted back to operator console 510.

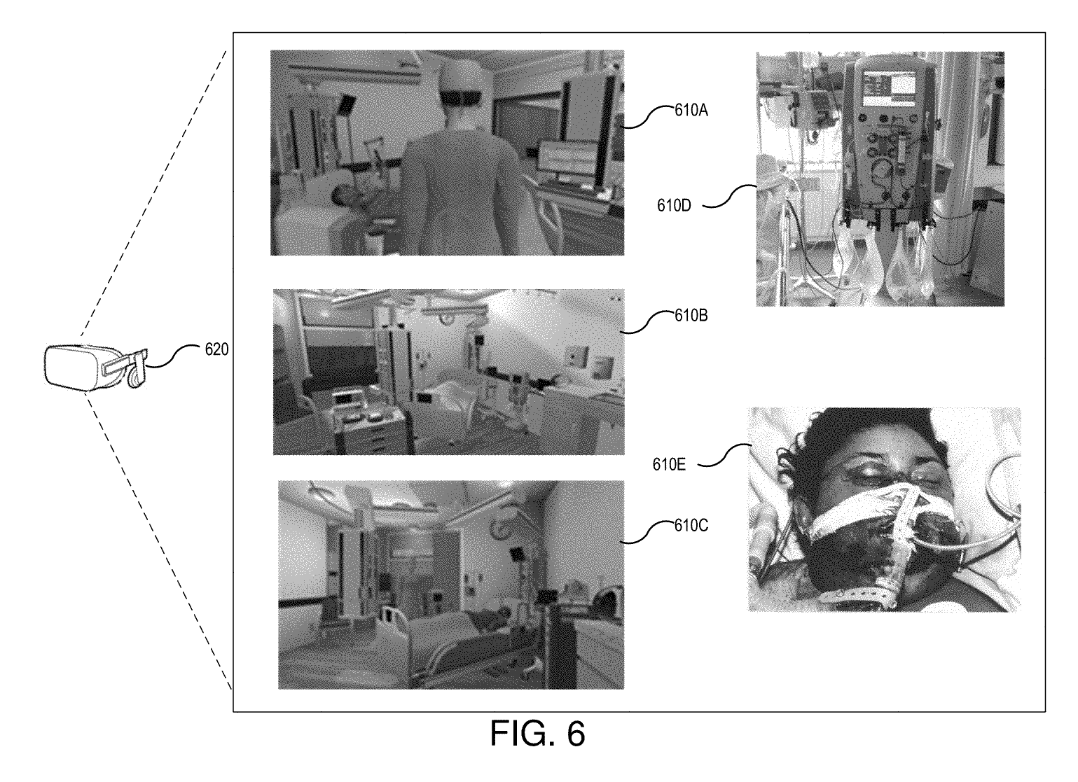

FIG. 6 illustrates sample images 610A-610E of participant view from a head mounted display (HMD) 620 in accordance with some embodiments of the present technology. These images may be presented to the user as part of a training scenario. For example, the scenario may include a 45 year old male patient is admitted with severe sepsis and presents with hypotension, respiratory failure and renal failure. Within 48 hours of admission, renal failure worsens requiring continuous renal replacement therapy (CRRT) and patient develops disseminated intravascular coagulation (DIC). The patient is sedated, on the ventilator, receiving vasopressor support for blood pressure and CRRT for renal failure. The images and scenes presented to the user may update and change based on the player's interactions (e.g., dialogue, interactions with medical equipment, etc.).

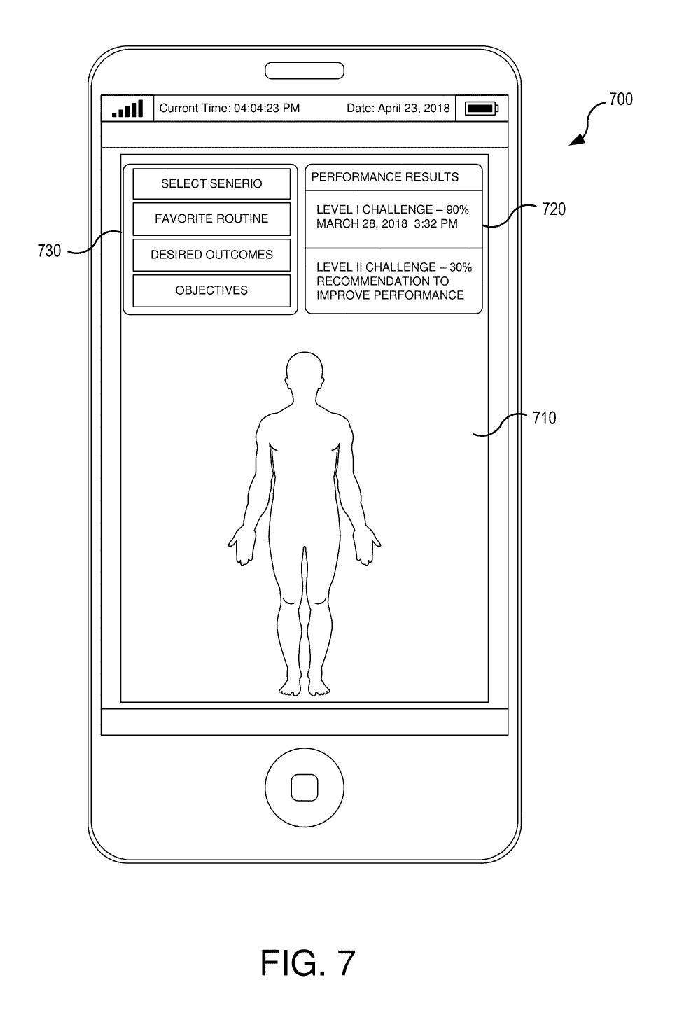

FIG. 7 illustrates an example of a graphical user interface 700 that may be used in one or more embodiments of the present technology. As illustrated in FIG. 7, the graphical user interface can include report area 710, performance area 720, and selection area 730. Report area 710 can provide various indications on the performance of players within the simulation. For example, if a training dummy has multiple areas that need to be addressed (e.g., leg and arm), then these areas may change colors based on the interactions from the simulation. For example, a first color may indicate that the player has yet to evaluate the area. When a player notes the area (e.g., with speech or touching of the training dummy), the report area 710 may change from a first color to a second color providing a visual indication to the operator that the area has been identified. Additional color changes may occur upon the player physically addressing the area.

Performance area 720 can provide overall performance results for the player and an indication of the challenge level. Selection area 730 can provide one or more interfaces allowing the operator to select various scenarios, outcome, objectives, and routines. For example, the operator may select an trauma virtual reality in an ICU hospital room. Single player game instruction may be selected. The operator may also set a specific set of objectives that need to be tracked.

At the beginning of the simulation, the player may be briefed providing some context for the current role. For example, the simulation may provide an indication to the player that the player's role in this experience is as an ICU Nurse. The overall mission can be set to explore the various aspects of this virtual environment that the player will be using as you move beyond this education module into the patient care experiences. There are tasks and objectives set by the operator and/or AI system may include one or more of the following: patient greetings, hand washing, supply cart interactives, bedside curtains, ventilator interactions, IV pump interactions, CRRT machine interactions, ICU bed interactions, may be selected.

For example, the system may monitor the patient greetings and award points for auditory statements of the following: state your name, role, and purpose for your presence in this setting as you enter the room. The meeting of each of these objectives teach the player how to talk to the avatar if we are going to use that later in the experience. The system may also monitor for hand washing at a sink. The player is expected to navigate to the sink, turn on the faucet for appropriate temperature (e.g., single handed or double handed), and begin the handwashing procedures. The player can earn points for turning on the faucet, holding hands under running water for five seconds or more, rubbing hands together for five seconds or more, getting soap by pressing the lever on the soap dispenser mounted to the right of the sink, learning how to wash between fingers, scrub, soap on for ten seconds, rinsing between fingers and hands for five seconds, obtaining paper towel from paper towel dispenser mounted to the wall to the left of the sink, drying appropriately for five seconds, using paper towel to turn off faucet, and disposing paper towel in garbage appropriately.

The system may also monitor the bedside supply cart interactions. For example, is the player able to pick up various items from the bedside supply cart. Ultimate challenge in this task may be to pull saline into a syringe. As such, the player may be expected to wipe the top of the saline bottle with an alcohol prep pad and let it dry. The player can earn points for picking up the syringe appropriately, taking the cap off appropriately, holding the syringe in dominant hand while picking bottle of saline up with nondominant hand, piercing top of saline bottle, holding saline bottle upside down and drawing saline into the syringe. The cap can rest on the supply cart until saline is filled in the syringe, recap syringe after saline is filled.

The bedside curtain objective may have multiple levels. For example, in some embodiments, there may be two levels to this experience. First, as the player enters the room and the curtain is already closed, the player may be expected to open it emergently. Second, the player may be working with the patient and need to close the curtain. As such, the system may award points (or otherwise score) on each level for maneuvering the curtain. The curtain may be a virtual curtain or a physical curtain within the training room having a fabric that is attached to the ceiling on a pulley track.

In accordance with various embodiments, the ventilator interaction may have multiple experience levels. First, the player may be expected to locate and move to the ventilator machine, find/point to the oxygen button, and/or push the button to administer 100% oxygen. The second experience level may include the player pointing to the in-line suctioning catheter, push the in-line catheter through the endotracheal tube (ETT) and into the patient's lungs, hold finger over suctioning mechanism as you pull the catheter out of the ETT. The third experience level may include the player pointing to the attachment point of the ventilator tubing and the endotracheal tube, pointing to the ambu bag, make sure oxygen tubing is attached to ambu bag, turning wall oxygen up to 100%, pointing to the attachment point of the ventilator tubing and the endotracheal tube, removing the ventilator tubing from the ETT, attaching the ambu bag with dominant hand, squeezing the ambu bag to deliver 100% oxygen.

Similarly, the IV pump interactions can include a player navigation over to the IV pole and point to the pump. The player may be expected to increase the rate of the IV fluids from 75 cc/hr to 125 cc/hr, push channel select button, rate button, use number pad to press 125, press start. The CRRT machine interactions may represent continuous renal replacement therapy. The player may be expected to navigate over to the CRRT machine and point to the machine, high return pressure alarm is sounding (this is the arterial side of the circuit, pressure gauge measures the positive pressure generated by the return pump, which sucks blood out of the filter and pushes it into the patient), manually visualize the circuit, checking for kinks, you find a kink in the line, unkink the line and press the continue button on the CRRT machine.

The ICU bed interactions may include the player moving to the side of the bed with the button panels. The player may be expected or asked to raise the head of the bed up so the patient is sitting up and then lower the head down for the patient to lie flat. The player may then be asked to lower the foot of the bed using the buttons. By finding the lever on the other side of the bed and stepping on it, the player may see a simulated response (e.g., raising or lowering of the bed).

Once the player has practiced all these tasks, the education level will present a ‘test run’ where the player must complete all of these items in a work flow (e.g., joined together in some instructional way), so that the player can practice moving from one behavior task to another with fluidity.

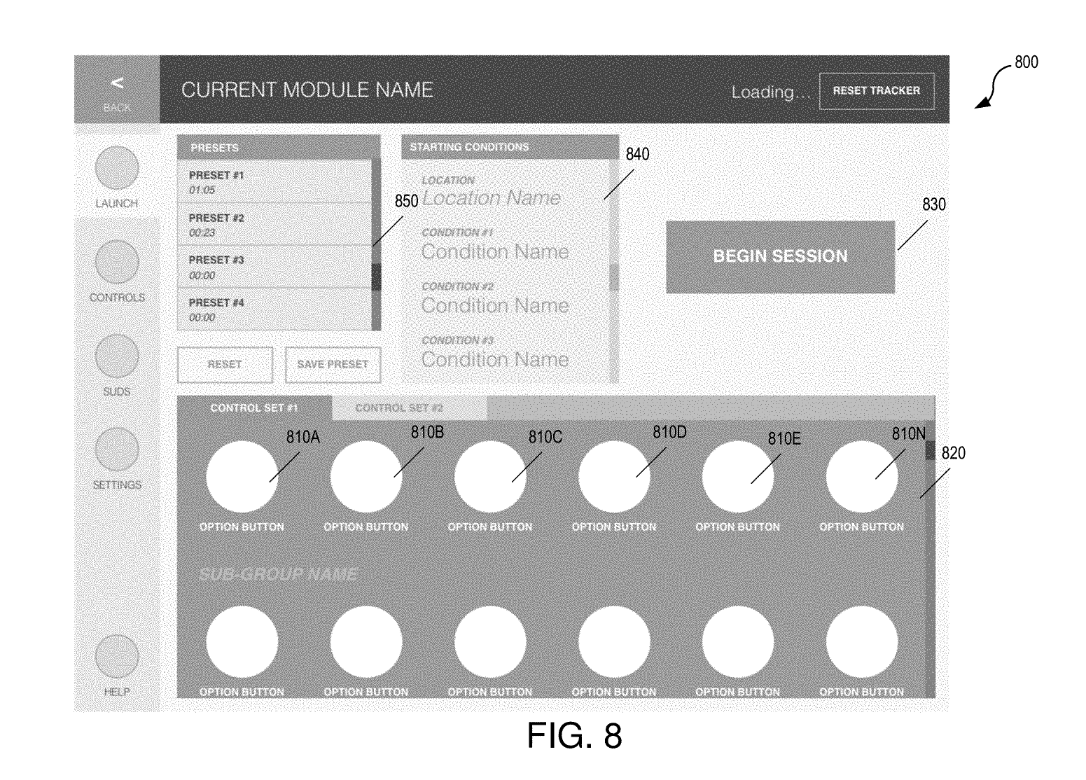

FIG. 8 illustrates an example of a graphical user interface 800 that may be part of a facilitator dashboard according to various embodiments of the present technology. The facilitator control dashboard 800 can be used to allow a VR administrator the ability to select various options, learning goals, specific training (e.g., on certain medical equipment, with certain procedures, etc.), and or other parameters for virtual reality scenario. In accordance with some embodiments, the options available may be specifically presented to the administrator or operator allowing them to choose (e.g., via button 810A-801N within option selection interface 820) one or more particular scenario options to launch in the VR environment. These options may be selected and set before the scenario begins (e.g., before session initiation button 830 is selected) or dynamically inserted as the virtual reality scenario unfolds.

Dashboard 800 may also provide a summary of specific starting conditions (e.g., location, role, etc.) within starting condition summary window 840. The administrator may be able to select each of the starting conditions. Upon selection, an interface containing the options for configuration (or reconfiguration) of the stating condition may be presented. The system may check for conflicts and only present options that will not conflict with other starting conditions or options selected by the administrator. In addition, various preset scenarios (or portions of scenarios) may be available for selection within preset window 850.

Dashboard 800 can also be used to replay specific sections of a scenario that may be distressing or triggering and launch tools to aid with building resilience techniques and to diffuse stressful responses to the scenarios. In some embodiments, dashboard 800 may include a complex 3D space 2D UI to facilitate the execution of certain decisions and tasks too ambiguous for VR simulation. In addition, in some embodiments, an augmented UI may be used for each interactive piece of equipment, allowing a higher degree of control where needed. The pre-existing assets can be animated (e.g., using both Maya modeling software and the Unity game engine). Participant sessions can be controlled via in-game prompts and facilitator control dashboard.

### Exemplary Computer System Overview

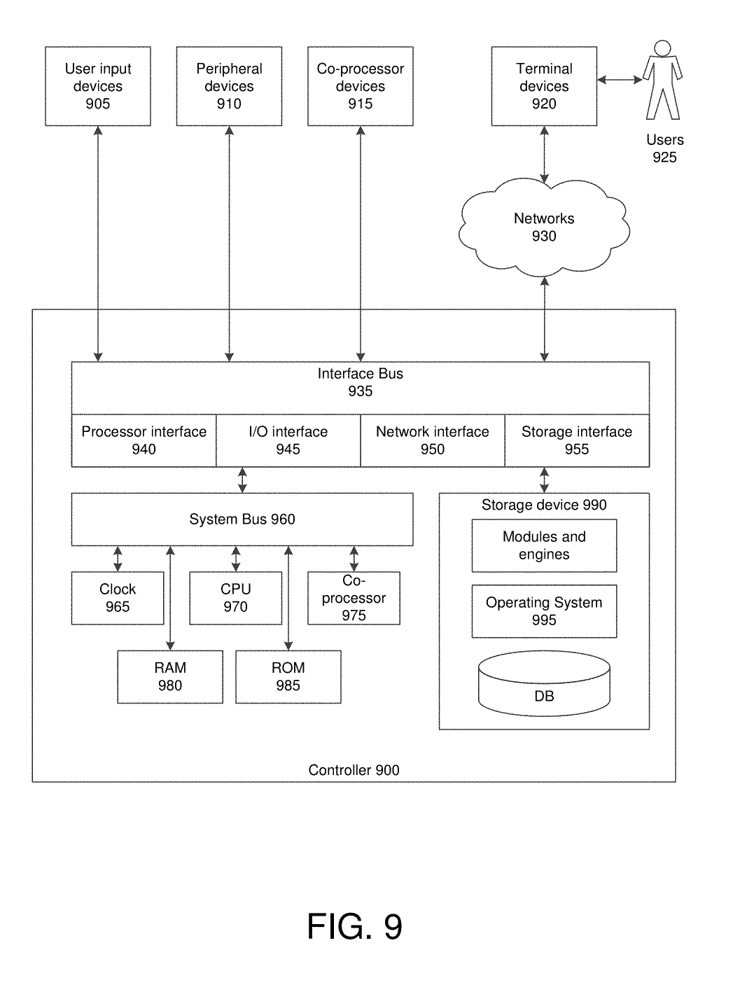

FIG. 9 is a block diagram illustrating an example machine representing the computer systemization of the simulation system that may be used in some embodiments of the present technology. A variety of these steps and operations may be performed by hardware components or may be embodied in computer-executable instructions, which may be used to cause a general-purpose or special-purpose processor (e.g., in a computer, server, or other computing device) programmed with the instructions to perform the steps or operations. For example, the steps or operations may be performed by a combination of hardware, software, and/or firmware.

The system controller 900 may be in communication with entities including one or more users 925 client/terminal devices 920 (e.g., devices 130A-130N, sensors 130E, etc.), user input devices 905, peripheral devices 910, an optional co-processor device(s) (e.g., cryptographic processor devices) 915, and networks 930. Users may engage with the controller 900 via terminal devices 920 over networks 930.

Computers may employ central processing unit (CPU) or processor to process information. Processors may include programmable general-purpose or special-purpose microprocessors, programmable controllers, application-specific integrated circuits (ASICs), programmable logic devices (PLDs), embedded components, combination of such devices and the like. Processors execute program components in response to user and/or system-generated requests. One or more of these components may be implemented in software, hardware or both hardware and software. Processors pass instructions (e.g., operational and data instructions) to enable various operations.

The controller 900 may include clock 965, CPU 970, memory such as read only memory (ROM) 985 and random access memory (RAM) 980 and co-processor 975 among others. These controller components may be connected to a system bus 960, and through the system bus 960 to an interface bus 935. Further, user input devices 905, peripheral devices 910, co-processor devices 915, and the like, may be connected through the interface bus 935 to the system bus 960. The interface bus 935 may be connected to a number of interface adapters such as processor interface 940, input output interfaces (I/O) 945, network interfaces 950, storage interfaces 955, and the like.

Processor interface 940 may facilitate communication between co-processor devices 915 and co-processor 975. In one implementation, processor interface 940 may expedite encryption and decryption of requests or data. Input output interfaces (I/O) 945 facilitate communication between user input devices 905, peripheral devices 910, co-processor devices 915, and/or the like and components of the controller 900 using protocols such as those for handling audio, data, video interface, wireless transceivers, or the like (e.g., Bluetooth, IEEE 1394a-b, serial, universal serial bus (USB), Digital Visual Interface (DVI), 802.11a/b/g/n/x, cellular, etc.). Network interfaces 950 may be in communication with the network 930. Through the network 930, the controller 900 may be accessible to remote terminal devices 920. Network interfaces 950 may use various wired and wireless connection protocols such as, direct connect, Ethernet, wireless connection such as IEEE 802.11a-x, and the like.

Examples of network 930 include the Internet, Local Area Network (LAN), Metropolitan Area Network (MAN), a Wide Area Network (WAN), wireless network (e.g., using Wireless Application Protocol WAP), a secured custom connection, and the like. The network interfaces 950 can include a firewall which can, in some aspects, govern and/or manage permission to access/proxy data in a computer network, and track varying levels of trust between different machines and/or applications. The firewall can be any number of modules having any combination of hardware and/or software components able to enforce a predetermined set of access rights between a particular set of machines and applications, machines and machines, and/or applications and applications, for example, to regulate the flow of traffic and resource sharing between these varying entities. The firewall may additionally manage and/or have access to an access control list which details permissions including, for example, the access and operation rights of an object by an individual, a machine, and/or an application, and the circumstances under which the permission rights stand. Other network security functions performed or included in the functions of the firewall, can be, for example, but are not limited to, intrusion-prevention, intrusion detection, next-generation firewall, personal firewall, etc., without deviating from the novel art of this disclosure.

Storage interfaces 955 may be in communication with a number of storage devices such as, storage devices 990, removable disc devices, and the like. The storage interfaces 955 may use various connection protocols such as Serial Advanced Technology Attachment (SATA), IEEE 1394, Ethernet, Universal Serial Bus (USB), and the like.

User input devices 905 and peripheral devices 910 may be connected to I/O interface 945 and potentially other interfaces, buses and/or components. User input devices 905 may include card readers, finger print readers, joysticks, keyboards, microphones, mouse, remote controls, retina readers, touch screens, sensors, and/or the like. Peripheral devices 910 may include antenna, audio devices (e.g., microphone, speakers, etc.), cameras, external processors, communication devices, radio frequency identifiers (RFIDs), scanners, printers, storage devices, transceivers, and/or the like. Co-processor devices 915 may be connected to the controller 900 through interface bus 935, and may include microcontrollers, processors, interfaces or other devices.

Computer executable instructions and data may be stored in memory (e.g., registers, cache memory, random access memory, flash, etc.) which is accessible by processors. These stored instruction codes (e.g., programs) may engage the processor components, motherboard and/or other system components to perform desired operations. The controller 900 may employ various forms of memory including on-chip CPU memory (e.g., registers), RAM 980, ROM 985, and storage devices 990. Storage devices 990 may employ any number of tangible, non-transitory storage devices or systems such as fixed or removable magnetic disk drive, an optical drive, solid state memory devices and other processor-readable storage media. Computer-executable instructions stored in the memory may include a platform having one or more program modules such as routines, programs, objects, components, data structures, and so on that perform particular tasks or implement particular abstract data types. For example, the memory may contain operating system (OS) component 995, modules and other components, database tables, and the like. These modules/components may be stored and accessed from the storage devices, including from external storage devices accessible through an interface bus.

The database components can store programs executed by the processor to process the stored data. The database components may be implemented in the form of a database that is relational, scalable and secure. Examples of such database include DB2, MySQL, Oracle, Sybase, and the like. Alternatively, the database may be implemented using various standard data-structures, such as an array, hash, list, stack, structured text file (e.g., XML), table, and/or the like. Such data-structures may be stored in memory and/or in structured files.

The controller 900 may be implemented in distributed computing environments, where tasks or modules are performed by remote processing devices, which are linked through a communications network, such as a Local Area Network (“LAN”), Wide Area Network (“WAN”), the Internet, and the like. In a distributed computing environment, program modules or subroutines may be located in both local and remote memory storage devices. Distributed computing may be employed to load balance and/or aggregate resources for processing. Alternatively, aspects of the controller 900 may be distributed electronically over the Internet or over other networks (including wireless networks). Those skilled in the relevant art(s) will recognize that portions of the system may reside on a server computer, while corresponding portions reside on a client computer. Data structures and transmission of data particular to aspects of the controller 900 are also encompassed within the scope of the disclosure.

### Conclusion

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof means any connection or coupling, either direct or indirect, between two or more elements; the coupling or connection between the elements can be physical, logical, or a combination thereof. Additionally, the words “herein,” “above,” “below,” and words of similar import, when used in this application, refer to this application as a whole and not to any particular portions of this application. Where the context permits, words in the above Detailed Description using the singular or plural number may also include the plural or singular number respectively. The word “or,” in reference to a list of two or more items, covers all of the following interpretations of the word: any of the items in the list, all of the items in the list, and any combination of the items in the list.

The above Detailed Description of examples of the technology is not intended to be exhaustive or to limit the technology to the precise form disclosed above. While specific examples for the technology are described above for illustrative purposes, various equivalent modifications are possible within the scope of the technology, as those skilled in the relevant art will recognize. For example, while processes or blocks are presented in a given order, alternative implementations may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed or implemented in parallel, or may be performed at different times. Further any specific numbers noted herein are only examples: alternative implementations may employ differing values or ranges.

The teachings of the technology provided herein can be applied to other systems, not necessarily the system described above. The elements and acts of the various examples described above can be combined to provide further implementations of the technology. Some alternative implementations of the technology may include not only additional elements to those implementations noted above, but also may include fewer elements.

These and other changes can be made to the technology in light of the above Detailed Description. While the above description describes certain examples of the technology, and describes the best mode contemplated, no matter how detailed the above appears in text, the technology can be practiced in many ways. Details of the system may vary considerably in its specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the above Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms. For example, while only one aspect of the technology is recited as a computer-readable medium claim, other aspects may likewise be embodied as a computer-readable medium claim, or in other forms, such as being embodied in a means-plus-function claim. Any claims intended to be treated under 35 U.S.C. § 112(f) will begin with the words “means for”, but use of the term “for” in any other context is not intended to invoke treatment under 35 U.S.C. § 112(f). Accordingly, the applicant reserves the right to pursue additional claims after filing this application to pursue such additional claim forms, in either this application or in a continuing application.

What is claimed is:

1. A method for operating a virtual reality environment, the method comprising:
generating, using an event simulator, a virtually reality scenario;
translating, using a control system, the virtual reality scenario into a set of commands for one or more devices,
wherein the one or more devices include a virtual reality headset to visually display the virtually reality scenario and a haptic glove to provide tactile feedback to a user of the virtual reality scenario; and
transmitting the set of commands to the one or more devices to generate the virtual reality scenario.

2. The method of claim 1, wherein the user and the one or more devices are within a training room having physical equipment that the user can interact with during the virtual reality scenario.

3. The method of claim 2, further comprising monitoring the physical equipment for interactions from the user.

4. The method of claim 2, further comprising:
receiving, from one or more sensors within the training room, indications of user responses to the virtual reality scenario; and
analyzing, using an artificial intelligence system, the indications of the user responses to generate updated scenes within the virtual reality scenario.

5. The method of claim 4, wherein the one or more sensors include one or more of a microphone to detect dialogue from the user, a camera to detect movement of the user, or heart rate monitor to record physiological response of the user to the virtual reality environment.

6. The method of claim 1, wherein the user is a first user of multiple users and the method further includes analyzing, using an artificial intelligence system, dialogue between the multiple users and updating scenes within the virtual reality scenario based on results from the artificial intelligence system analysis.

7. A system comprising:
a training room having physical equipment and sensors capable of monitoring and recording interactions from a user;
a database of user scenarios, wherein the user scenarios include dialogue between individuals, medical equipment parameters, and physiological parameters of a patient;
an artificial intelligence system capable of ingesting the user scenarios stored in the database, and upon receiving input signals from the physical equipment and sensors within the training room generating updates to a dynamically changing virtual reality scenario;
a virtual reality event simulator configured to receive the updates to the dynamically changing virtual reality scenario from the artificial intelligence system and generate a sequence of scenes to be presented to the user; and
a control system to receive the updates to the dynamically changing virtual reality scenario from the artificial intelligence system and the scenes from the virtual reality event simulator and translate the updates to commands to control the physical equipment and sensors to create a unique experience for the user.

8. The system of claim 7, wherein the user is using a virtual reality headset and the control system generates updated images that can be presented to the user via the virtual reality headset.

9. The system of claim 7, further comprising a scent synthesizer to generate one or more smells as indicated by the control system.

10. The system of claim 9, wherein the one or more smells include a smell of blood.

11. The system of claim 7, further comprising an operator console allowing an operator to select one or more training objectives to be presented to the user.

12. The system of claim 7, wherein the physical equipment includes a dummy, a bed, one or more IV systems, a curtain, a sink, and a ventilator machine.

13. The system of claim 7, wherein the physical equipment or sensors include one or more of a microphone to detect dialogue from the user or a camera to detect movement of the user.

14. The system of claim 7, further comprising a virtual reality headset configured to receive the sequence of scenes generated by the virtual reality event simulator and present the sequence of scenes to the user.

15. The system of claim 7, further comprising one or more speakers to present sounds as part of the dynamically changing virtual reality scenario.

16. The system of claim 15, wherein the sounds include dialogue from other characters, patient noises, physical machine noises, or hospital announcements.

17. A non-transitory computer-readable storage medium containing a set of instructions that when executed by one or more processors cause a machine to:
generate a virtually reality scenario;
translate the virtual reality scenario into a set of commands for one or more devices,
wherein the one or more devices include a virtual reality headset to visually display the virtually reality scenario and a haptic glove to provide tactile feedback to a user of the virtual reality scenario; and
transmit the set of commands to the one or more devices to generate the virtual reality scenario.

18. The non-transitory computer-readable storage medium of claim 17, wherein the user and the one or more devices are within a training room having physical equipment that the user can interact with during the virtual reality scenario.

19. The non-transitory computer-readable storage medium of claim 17, wherein the set of instructions when executed by the one or more processors further cause the machine to ingest a plurality of scenarios stored in a database and identify, using an artificial intelligence system, updates to the virtual reality scenario.

20. The non-transitory computer-readable storage medium of claim 17, herein the set of instructions when executed by the one or more processors further cause the machine to analyze interactions of the user throughout the virtual reality scenario and generate a training plan specific to that user.
